(19) United States
(12) Patent Application Publication
(10) Pub. No.: US 2002/0068861 A1
(43) Pub. Date: Jun. 6, 2002

(54) Structure of an electrode plate penetrable by X-rays

(76) Inventor: Wen Yang, Taipei (TW)

Correspondence Address: Wen Yang, PO BOX 82-144, TAIPEI, TW

(21) Appl. No.: 09/726,355

(22) Filed: Dec. 1, 2000

(51) Int. Cl. A61B 5/04

(52) U.S. Cl. 600/382

(57) ABSTRACT

An electrode plate includes a button made of conductive rubber and having an upper side on which there is a tubular protuberance having a closed upper end and an open lower end, a label having a center hole receiving the protuberance of the button and adhered to an upper side of the button, a circular adhesive member adhered to a lower side of the button, and an adhesive sheet adhered to a lower side of the label, whereby the improved electrode plate according to the present invention not only improves the complicated procedures for manufacturing electrode plates, but can also prevent spots on X-ray film, thereby ensuring the accuracy of X-ray film

### BACKGROUND OF THE INVENTION

1. Technical Field of the Invention

This invention is related to an electrode plate and in particular to an improvement in the structure of an electrode plate which can be penetrated by X-rays.

2. Description of the Prior Art

In present society, the use of medical services is increasing day-by-day, and most of the medical materials used are becoming disposable. The advantage of disposable medical materials is that their quality and sanitary control can be retained. However, disposable medical materials must be low in price in order to decrease medical costs.

Disposable electrode plates are widely used in the medical field for inspecting cardiograph instruments, for giving emergency electric shocks, electric knife surgery, or MRI. However, the electrode plates are specifically designed for a single purpose, so that a different type of electrode plate must be used for a different task, thereby limiting the utilization of electrode plates.

Referring to FIGS. 1, 2 and 3, the conventional electrode plate generally comprises a metallic upper button 10, a metallic lower button 11 engageable with the metallic upper button 10, a label 12 arranged between the metallic upper and lower buttons 10 and 11, an adhesive member 13, and a circular member 14.

For this kind of electrode plate, it is necessary to remove the electrode plate before taking x-rays, otherwise there will be spots on the film produced by the x-ray. Then it is necessary to stick the electrode plate on the body again for the next inspection procedure. Generally, the number of electrode plates used in such a procedure are eight to ten pieces. This causes much inconvenience to the medical practitioner.

Another conventional electrode plate is made of an integral plastic member. Although x-rays can penetrate such kind of electrode plate, the outer surface of the plastic body must be electroplated with a layer of silver/silver chloride. However, the electroplating process will pollute the environment and because the outer side is coated with silver chloride which is an active chemical substance, its contact with instruments will cause corrosion, so that after a certain period of time, the metal input end of the instrument will be corroded, thereby causing instability of current signal readings, and causing mistakes to be made in signal readings.

### SUMMARY OF THE INVENTION

This invention is related to an electrode plate and in particular to an improvement in the structure of an electrode plate which can be penetrated by X-rays.

It is the primary object of the present invention to provide an electrode plate includes a button made of conductive rubber and having an upper side on which there is a tubular protuberance having a closed upper end and an open lower end, a label having a center hole receiving the protuberance of the button and adhered to an upper side of the button, a circular adhesive member adhered to a lower side of the button, and an adhesive sheet adhered to a lower side of the label, whereby the improved electrode plate according to the present invention not only improves the complicated procedures for manufacturing electrode plates, but can also prevent spots on X-ray film, thereby ensuring the accuracy of X-ray film.

The foregoing object and summary provide only a brief introduction to the present invention. To fully appreciate these and other objects of the present invention as well as the invention itself, all of which will become apparent to those skilled in the art, the following detailed description of the invention and the claims should be read in conjunction with the accompanying drawings. Throughout the specification and drawings identical reference numerals refer to identical or similar parts. Many other advantages and features of the present invention will become manifest to those versed in the art upon making reference to the detailed description and the accompanying sheets of drawings in which a preferred structural embodiment incorporating the principles of the present invention is shown by way of illustrative example.

### BRIEF DESCRIPTION OF THE DRAWINGS

FIG. 1 is an exploded view of a prior art electrode plate;

FIG. 2 is a sectional view of the prior art electrode plate;

FIG. 3 is a perspective view of the prior art electrode plate;

FIG. 4 is an exploded view of the present invention;

FIG. 5A illustrates the top of the present invention;

FIG. 5B illustrates the bottom of the present invention;

FIG. 6 is a sectional view of the present invention;

FIG. 7 is a perspective view of the present invention; and

FIG. 8 illustrates the connection of the present invention with an electrocardiograph.

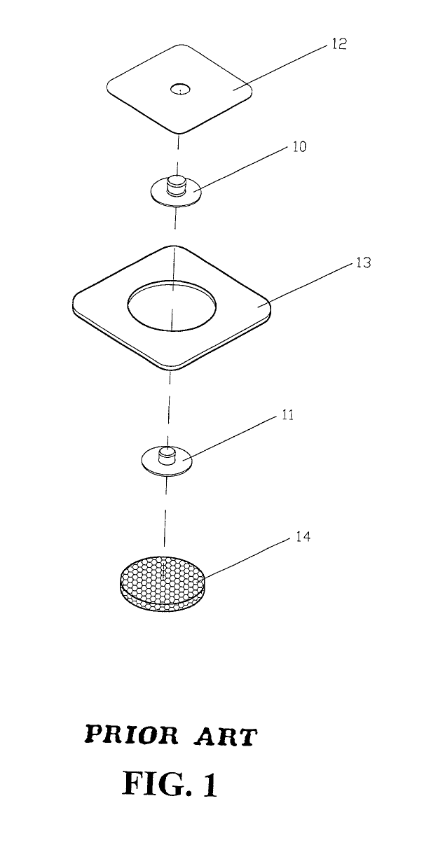

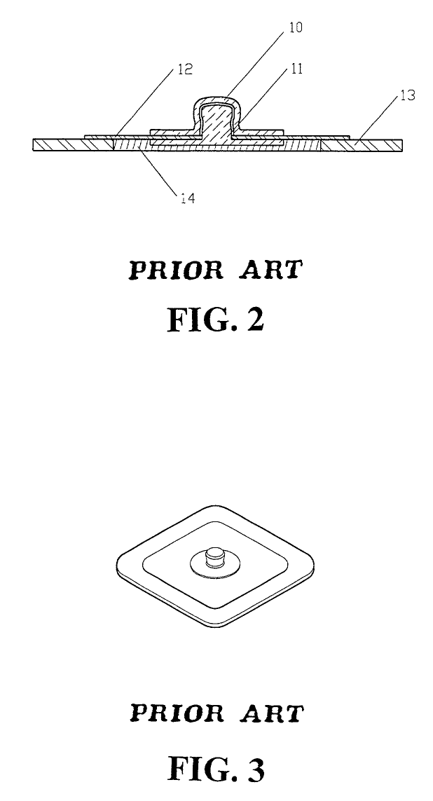

### DETAILED DESCRIPTION OF THE PREFERRED EMBODIMENT

For the purpose of promoting an understanding of the principles of the invention, reference will now be made to the embodiment illustrated in the drawings. Specific language will be used to describe same. It will, nevertheless, be understood that no limitation of the scope of the invention is thereby intended, such alterations and further modifications in the illustrated device, and such further applications of the principles of the invention as illustrated herein being contemplated as would normally occur to one skilled in the art to which the invention relates.

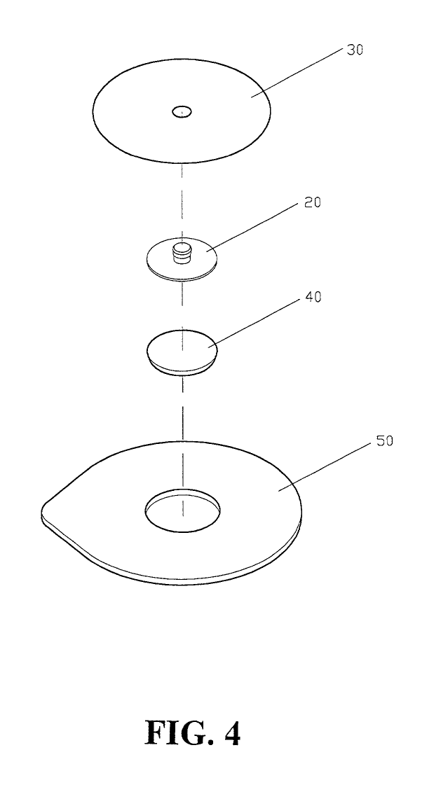

With reference to the drawings and in particular to FIGS. 4, 5A, and 6 thereof, the electrode plate according to the present invention generally comprises a thin circular label 30, a button 20 made of conductive rubber, a thin circular adhesive member 40, and an adhesive sheet 50. The thin circular label 30 has a circular hole at the center and has an adhesive bottom The button 20 has a tubular protuberance at the center of its top which extends upwardly through the circular hole of the thin circular label 30, so that the upper side of the button 20 is adhered to the bottom of the thin circular label 30. The thin circular adhesive member 40 is stuck to the bottom side of the button 20. The adhesive sheet 50 has a center through opening. The label 30 together with the button 10 and the adhesive member 40 is adhered on the top of the adhesive sheet 50.

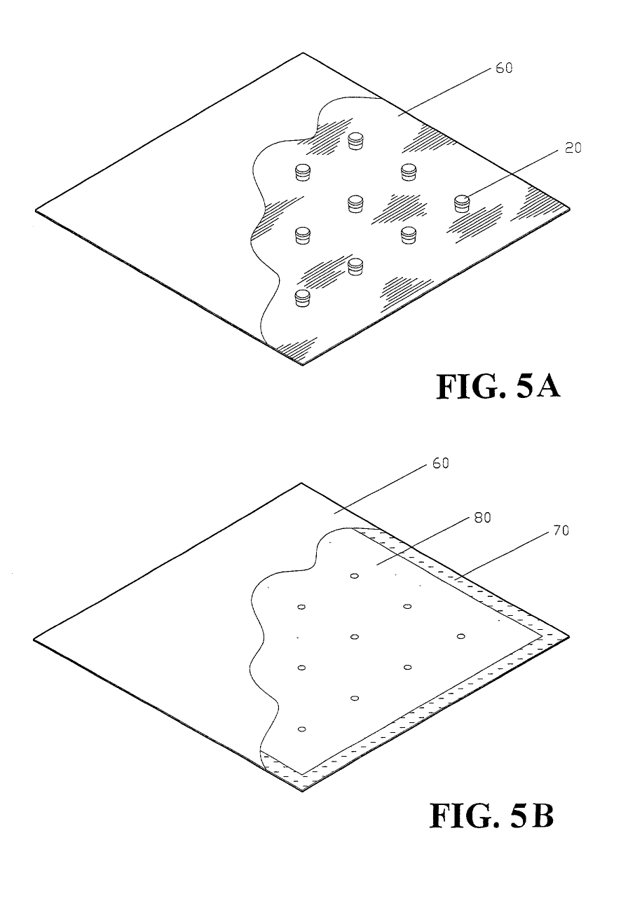

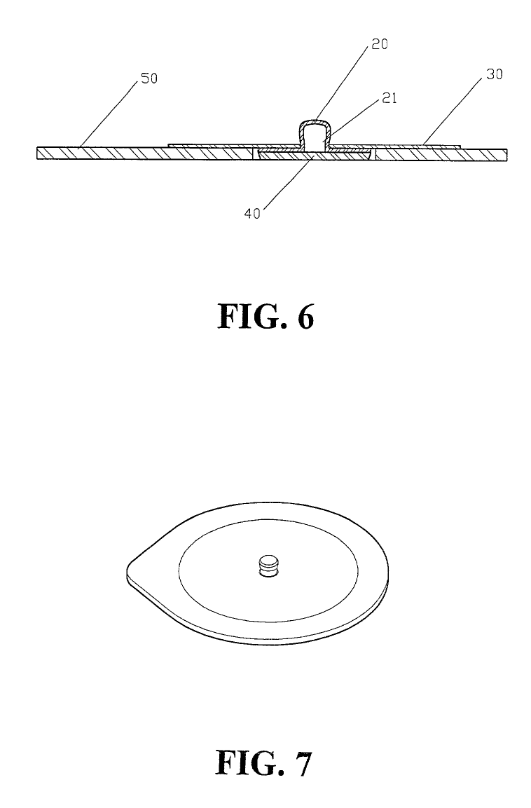

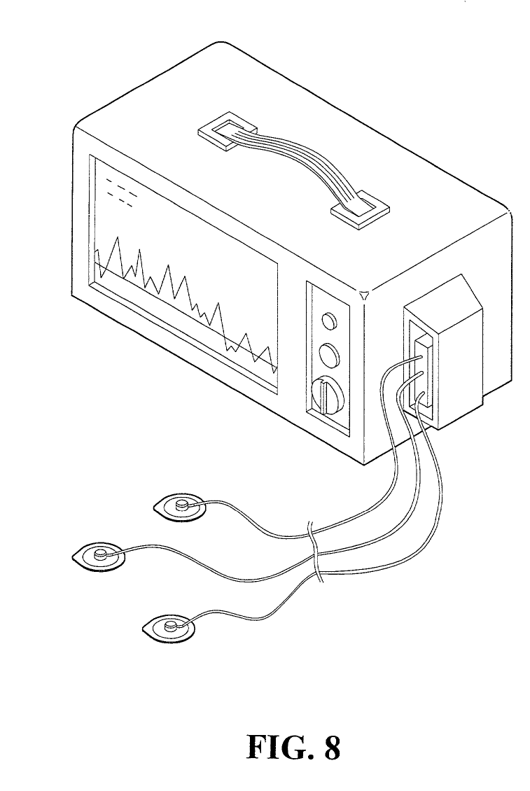

As shown in FIG. 6, the button 20 is made of conductive rubber 60 and has a tubular protuberance 21 which is closed at the upper end and open at the lower end. The bottom of the button 20 is coated with a layer of silver/silver chloride 70 (see FIGS. 5A and 5B) which is two times as thick as the layer of silver/silver chloride coated on a conventional electrode plate, so that the transmission of cardiograph signal of the button according to the present invention will be more stable than the conventional metallic button thereby decreasing the spots produced in X-ray film and preventing inaccuracy.

As shown in FIGS. 5A and 5B, the buttons 20 are formed from a sheet of conductive rubber by a die press and will be the same shape as the conventional ones. Then, the bottom of the button is coated with a layer 70 of silver/silver chloride thereby forming a contact surface 80.

The silver/silver chloride treatment can detect the correct current signal without causing damage to the skin of a human being.

In conclusion, the improved electrode plate according to the present invention not only improves the complicated procedures for manufacturing electrode plates, but can also prevent spots on X-ray film, thereby ensuring the accuracy of X-ray film.

It will be understood that each of the elements described above, or two or more together may also find a useful application in other types of methods differing from the type described above.

While certain novel features of this invention have been shown and described and are pointed out in the annexed claim, it is not intended to be limited to the details above, since it will be understood that various omissions, modifications, substitutions and changes in the forms and details of the device illustrated and in its operation can be made by those skilled in the art without departing in any way from the spirit of the present invention.

I claim:

1. An electrode plate comprising:
a button made of conductive rubber and having an upper side on which there is a tubular protuberance having a closed upper end and an open lower end;
a label having a center hole receiving said protuberance of said button and adhered to an upper side of said button;
a circular adhesive member adhered to a lower side of said button; and
an adhesive sheet adhered to a lower side of said label.

2. The electrode plate as claimed in claim 1, wherein said button has a bottom coated with a layer of silver chloride.
